(19) United States
(12) Patent Application Publication
(10) Pub. No.: US 2003/0027773 A1
(43) Pub. Date: Feb. 6, 2003

(54) PROTEIN-POLYCATION CONJUGATES

(76) Inventors: MAX L. BIRNSTIEL, WIEN (AT); MATTHEW COTTEN, WIEN (AT); ERNST WAGNER, LANGENZERSDORF (AT)

Correspondence Address: STERNE, KESSLER, GOLDSTEIN & FOX PLLC, 1100 NEW YORK AVENUE, N.W., SUITE 600, WASHINGTON, DC, 20005-3934, US

(21) Appl. No.: 08/098,268

(22) PCT Filed: Mar. 24, 1992
(86) PCT No.: PCT/EP92/00642

(30) Foreign Application Priority Data
Mar. 24, 1992 (US) PCT/EP92/00642
Mar. 29, 1991 (DE) P 41 10 409.9

(51) Int. Cl. A61K 48/00; C07K 16/40; C07H 21/04; C12N 15/87

(52) U.S. Cl. 514/44; 514/2; 514/12; 530/387.1; 530/388.1; 530/358; 530/324; 435/455; 536/23.1; 536/24.5

(57) ABSTRACT

New protein-polycation conjugates which are capable of forming soluble complexes with nucleic acids contain as their protein component an antibody directed against a cell surface protein, with the ability to bind to the cell surface protein so that the complexes formed are absorbed into cells which express the cell surface protein and are expressed therein. Complexes for use in pharmaceutical preparations contain a therapeutically or gene therapeutically active nucleic acid.

The invention relates to new protein-polycation conjugates for transporting compounds having an affinity for polycations, particularly nucleic acids, into human or animal cells.

In recent years, nucleic acids have acquired greater significance as therapeutically active substances.

Antisense RNAs and DNAs have proved to be effective agents for selectively inhibiting certain genetic sequences. Their mode of activity enables them to be used as therapeutic agents for blocking the expression of certain genes (such as deregulated oncogenes or viral genes) in vivo. It has already been shown that short antisense oligonucleotides can be imported into cells and perform their inhibiting activity therein (Zamecnik et al., 1986), even though the intracellular concentration thereof is low, partly because of their restricted uptake through the cell membrane owing to the strong negative charge of the nucleic acids.

Another method of selectively inhibiting genes consists in the application of ribozymes. Here again there is the need to guarantee the highest possible concentration of active ribozymes in the cell, for which transportation into the cell is one of the limiting factors.

Numerous solutions have already been proposed for improving the transportation of nucleic acids into living cells, which is one of the limiting factors in the therapeutic use thereof.

One of the known approaches to solving the problem of conveying inhibiting nucleic acid into the cell consists in direct modification of the nucleic acids, e.g. by substituting the charged phosphodiester groups with uncharged groups. Another possible method of direct modification consists in the use of nucleoside analogues. However, these proposals have various disadvantages, e.g. reduced binding to the target molecule, a poorer inhibitory effect and possible toxicity.

There is also a particular need for an efficient system for introducing nucleic acid into living cells in gene therapy. In this, genes are locked into cells in order to achieve in vivo the synthesis of therapeutically active gene products, e.g. in order to replace the defective gene in the case of a genetic defect. “Classic” gene therapy is based on the principle of achieving a long term cure by means of a single treatment. However, there is also a need for treatment methods in which the therapeutically active DNA (or mRNA) can be used as a drug (“gene therapeutic agent”) which is administered once or repeatedly, as necessary. Examples of genetically caused diseases in which gene therapy represents a promising approach are haemophilia, beta-thalassaemia and “Severe Combined Immune Deficiency” (SCID), a syndrome caused by a genetically induced deficiency of the enzyme adenosine deaminase. Other possible applications are in immune regulation, in which the administration of functional nucleic acid which codes for a secreted protein antigen or for an unsecreted protein antigen achieves a humoral or intracellular immunity by means of vaccination. Other examples of genetic defects in which administration of nucleic acid which codes for the defective gene can be given, for example, in a form individually tailored to the particular requirements include muscular dystrophy (dystrophin gene), cystic fibrosis (cystic fibrosis conductance regulator gene), hypercholesterolaemia (LDL receptor gene). Gene therapeutic methods of treatment are also potentially of significance when hormones, growth factors or proteins with a cytotoxic or immunomodulating effect are to be synthesised in the body.

The technologies which have hitherto progressed furthest for the use of nucleic acids in gene therapy make use of retroviral systems for the transfer of genes into the cell (Wilson et al., 1990, Kasid et al., 1990). The use of retroviruses does, however, present problems because it involves, at least in a small percentage, the danger of side effects such as infection with the virus (by recombination with endogenous viruses and possible subsequent mutation into the pathogenic form) or by formation of cancer. Moreover, the stable transformation of the somatic cells of the patient as achieved by means of retroviruses is not desirable in every case since it may only make the treatment more difficult to reverse, e.g. if side effects occur.

There has therefore been a search for alternative methods of enabling the expression of non-replicating DNA in the cells.

There are various known techniques for the genetic transformation of mammalian cells in vitro, but their use in vivo is restricted (they include the introduction of DNA by means of liposomes, electroporation, microinjection, cell fusion, DEAE-dextran or the calcium phosphate precipitation method).

Recent efforts to develop methods for in vivo gene transfer have concentrated on the use of the cationic lipid reagent lipofectin; a plasmid injected by means of this reagent has been shown to be capable of being expressed in the body (Nabel et al., 1990).

Another recently developed method uses microparticles of tungsten or gold onto which DNA has been absorbed, by means of which the cells can be bombarded with high energy (Johnston, 1990, Yang et al., 1990). Expression of the DNA has been demonstrated in various tissues.

A soluble system which can be used in vivo to convey the DNA into the cells in targeted manner was developed for hepatocytes and is based on the principle of coupling polylysine to a glycoprotein to which a receptor provided on the hepatocyte surface responds and then, by adding DNA, forming a soluble glycoprotein/polylysine/DNA complex which is absorbed into the cell and, once absorbed, allows the DNA sequence to be expressed (Wu and Wu, 1987).

This system is specific to hepatocytes and is defined, in terms of its function, by the relatively well characterised absorption mechanism by means of the asialoglycoprotein receptor.

A broadly applicable and efficient transport system makes use of the transferrin receptor for absorbing nucleic acids into the cell by means of transferrin-polycation conjugates. This system is the subject of European Patent Application A1 388 758. It was shown that transferrin-polycation/DNA complexes are efficiently absorbed and internalised in living cells, using as the polycation component of the complexes polylysine of various degrees of polymerisation and protamine. Using this system, inter alia, a ribozyme gene inhibiting the erbB-oncogene was introduced into erbB-transformed hen cells and the erbB inhibiting effect was demonstrated.

The aim of the present invention was to prepare a system by means of which the selective transport of nucleic acids into higher eukaryotic cells is possible.

It was surprisingly found that antibodies which bind to a cell surface protein can be used for transporting nucleic acids into higher eukaryotic cells if they are conjugated with polycations.

It has been shown that the cell surface protein CD4 used by the HIV virus during infection can be used to transport nucleic acid into the cell by complexing the nucleic acid which is to be imported with a protein/polycation conjugate the protein content of which is an antibody directed against CD4, and by contacting CD4-expressing cells with the resulting protein-polycation/DNA complexes.

It has also been demonstrated within the scope of the present invention that by means of antibody-polycation conjugates containing an antibody against CD7, DNA is introduced into cells of the T-cell lineage and expressed in these cells. (CD7 is a cell surface protein with an as yet unknown physiological role which has been detected on thymocytes and mature T-cells. CD7 is a reliable marker for acute T-cell leukaemia (Aruffo and Seed, 1987)).

For antibody-polycation conjugates which contained an antibody directed against the membrane fraction of rat pancreas carcinoma cells it was also shown that DNA which is complexed with the conjugates is introduced into such cells and expressed therein.

Within the scope of the present invention it was thus demonstrated by means of antibody-polycation conjugates with various antibody components that, with the aid of such conjugates in cells which express the particular surface antigen against which the antibody is directed, the internalisation and expression of DNA can be achieved.

The invention thus relates to new protein-polycation conjugates which are capable of forming complexes with nucleic acids, the protein component being an antibody against a cell surface protein which is capable of binding to the cell surface protein, so that the complexes formed are absorbed into the cells which express the cell surface protein.

Hereinafter, antibodies against cell surface proteins of the target cells are referred to as “antibodies”.

The invention further relates to antibody-polycation/nucleic acid complexes in which the conjugates according to the invention are complexed with a nucleic acid which is to be transported into the target cells which express the cell surface antigen against which the antibody is directed.

Within the scope of the present invention it has been shown that DNA as a component of the complexes according to the invention is efficiently absorbed into and expressed in cells which express the particular antigen against which the antibodies are directed, the uptake of DNA into the cell increasing as the conjugate content increases.

Suitable antibodies are all those antibodies, particularly monoclonal antibodies, against cell surface antigens or the fragments thereof which bind to the cell surface antigen, e.g. Fab′ fragments (Pelchen-Matthews et al., 1989).

Instead of conventional monoclonal antibodies or fragments thereof it is possible to use antibody variants or sections consisting of a combination of segments of the heavy and light chain or possibly of the heavy chain on its own. The preparation of such “alternative” antibodies by cloning by means of polymerase chain reaction and expression in E.coli have been briefly described (Sastry et al., 1989; Orlandi et al., 1989; Chaudhary et al., 1990). In order to avoid immune responses when used therapeutically in humans, particularly for in vivo treatment over a long period of time, humanised antibodies (e.g. Co and Queen, 1991) or human antibodies are preferred for such applications. A survey of monoclonal antibodies and antibody variants produced by genetic engineering is provided by Waldmann, 1991. Antibodies against surface molecules on human leukocytes are mentioned by Knapp et al., 1989.

The choice of the antibody is determined particularly by the target cells, e.g. by certain surface antigens or receptors which are specific or largely specific to one type of cell and thus enable a directed introduction of nucleic acid into this type of cell.

The conjugates according to the invention permit narrower or wider selectivity with regard to the cells to be treated with nucleic acid, depending on the surface antigen against which the antibody contained in the conjugate is directed, and enable the flexible use of therapeutically or gene therapeutically active nucleic acid.

Within the scope of the present invention, the conjugate component may consist of antibodies or fragments thereof which bind to the cell, as a result of which the conjugate/DNA complexes are internalised, particularly by endocytosis, or antibody (fragments) the binding/internalising of which is carried out by fusion with cell membrane elements.

What is essential for the suitability of antibodies (antibody fragments) within the scope of the invention is that

a) they should be recognised by the specific type of cell into which the nucleic acid is to be introduced and that their binding capacity is unaffected or not substantially affected if they are conjugated with the polycation, and

b) that within the scope of this property they are capable of carrying nucleic acid “piggyback” into the cell by the route which they use.

With the proviso that they satisfy the conditions set out under a) and b), it is theoretically possible to use any antibodies directed against surface antigens for the purposes of the present invention. These include antibodies against cell surface proteins which are specifically expressed on a certain type of cell, e.g. when the invention is applied to cells of the T-cell lineage, antibodies against the CD4 or CD7 antigens which are characteristic of this type of cell.

Other antibodies which are suitable for the purposes of the invention are antibodies against receptors which come under the definition “cell surface proteins” within the scope of the invention. Examples of receptors are the transferrin receptor, the hepatocyte-asialoglycoprotein receptor, receptors for hormones or growth factors (insulin, EGF-receptor), receptors for cytotoxically active substances such as TNF or receptors which bind the extracellular matrix, such as the fibronectin receptor or the vitronectin receptor. Also suitable are antibodies against ligands for cell surface receptors provided that they do not affect the ability of the ligand to bind to its receptor.

For targeted use on tumour cells it is particularly suitable to use antibodies against specific cell surface proteins expressed on the tumour cells in question, so-called tumour markers.

Polycations which are suitable according to the invention include, for example, homologous polycations such as polylysine, polyarginine, polyornithine or heterologous polycations having two or more different positively charged amino acids, these polycations possibly having different chain lengths, as well as non-peptide synthetic polycations such as polyethyleneimine. Other suitable polycations are natural DNA-binding proteins of a polycationic nature such as histones or protamines or analogues or fragments thereof.

The size of the polycations is not critical; in the case of polylysine it is preferably such that the sum of the positive charges is about 20 to 1000 and is matched to the particular nucleic acid to be transported. For a given length of nucleic acid the length of the polycation is not critical. If for example the DNA has 6,000 bp and 12,000 negative charges, the quantity of polycation is, for example, 60 mol polylysine 200 or 30 mol polylysine 400 or 120 mol polylysine 100, etc. The average person skilled in the art is also capable of choosing other combinations of polycation sizes and molar quantities by means of routine experiments which are easy to carry out.

The antibody polycation conjugates according to the invention may be prepared chemically in a method known for the coupling of peptides, and if necessary the individual components may be provided before the coupling reaction with linker substances (this measure is necessary if there is no available functional group suitable for coupling such as a mercapto or alcohol group. The linker substances are bifunctional compounds which are reacted first with functional groups of the individual components, after which the modified individual components are coupled.

Depending on the desired properties of the conjugates, particularly with respect to their stability, coupling may be carried out by

a) Disulphide bridges which can be cleaved again under reducing conditions (e.g. using succinimidyl-pyridyldithiopropionate (Jung et al., 1981).

b) Using compounds which are largely stable under biological conditions (e.g. thioethers by reacting maleimido linkers with sulfhydryl groups of the linker bound to the second component).

c) Bridges which are unstable under biological conditions, e.g. ester bonds, or acetal or ketal bonds which are unstable under slightly acidic conditions.

When using the antibodies which are produced by genetic engineering as mentioned above it is also possible to prepare the conjugates according to the invention by the recombinant method, which has the advantage of making it possible to obtain accurately defined and unified compounds, whereas chemical coupling initially produces mixtures of conjugate which have to be separated.

The recombinant preparation of the conjugates according to the invention may be carried out using methods known for the preparation of chimeric polypeptides. The polycationic peptides may vary in their size and amino acid sequence. Production by genetic engineering also has the advantage of allowing modification of the antibody part of the conjugate, for example by increasing the ability to bind to the cell surface protein, by suitable mutation, or by using an antibody component which has been shortened to that part of the molecule which is responsible for binding to the cell surface protein. It is particularly appropriate for recombinant production of the conjugates according to the invention to use a vector which contains the sequence coding for the antibody component, as well as a polylinker into which the required sequence coding for the polycationic peptide has been inserted. In this way it is possible to obtain a set of expression plasmids from which the plasmid containing the desired sequence can be selected to be mused as necessary for the expression of the conjugate according to the invention.

If the antibody contains suitable carbohydrate chains, it may be linked to the polycation via one or more of these carbohydrate chains. Conjugates of this kind have the advantage, over conjugates prepared by conventional coupling methods, that they are free from modifications originating from the linker substances used. A suitable method of preparing glycoprotein-polycation conjugates is disclosed in German Patent Application P 41 15 038.4; it was briefly described by Wagner et al., 1991.

The molar ratio of antibody to polycation is preferably 10:1 to 1:10, although it should be borne in mind that aggregates may be formed. However, this ratio may if necessary be within wider limits provided that the condition is met that complexing of the nucleic acid or acids takes place and it is ensured that the complex formed is bound to the cell surface protein and conveyed into the cell. This can be checked by simple tests carried out in each individual case, e.g. by bringing cell lines which express the cell surface antigen into contact with the complexes according to the invention and then investigating them for the presence of nucleic acid or the gene product in the cell, e.g. by Southern blot analysis, hybridisation with radioactively labelled complementary nucleic acid molecules, by amplification using PCR or by detecting the gene product of a reporter gene.

For specific applications, particularly for screening in order to find suitable antibodies, it may be advantageous not to couple the antibody directly to the polycation: for efficient chemical coupling it is generally necessary to use a larger amount (more than 1 mg) of starting antibody and furthermore the coupling may optionally deactivate the antibody binding domain. To get round this problem and allow rapid screening of suitable antibodies it is first of all possible to prepare a protein A polycation conjugate to which the antibody is subsequently bound, optionally in a form already complexed with nucleic acid, just before the transfection of the cells, by means of the F_{c}-binding domain of protein A (Surolia et al., 1982). The nucleic acid complexes formed with the protein A conjugates allow rapid testing of antibodies for their suitability for importing nucleic acid into the particular type of cells to be treated. The coupling of protein A with the relevant polycation is carried out analogously to the direct coupling with the antibody. When protein A-antibody-polycation conjugates are used it may be advantageous first to incubate the cells which are to be treated with the antibody, to free the cells from excess antibody and then treat them with the protein A-polycation/nucleic acid complex. Optionally, protein A is modified, e.g. by amounts of protein G, in order to increase its affinity for the antibodies.

The nucleic acids to be transported into the cell may be DNAs or RNAs, there being no restrictions on the nucleotide sequence. The nucleic acids may be modified provided that the modification does not affect the polyanionic nature of the nucleic acids; these modifications include, for example, the substitution of the phosphodiester group by phosphorothioates or the use of nucleoside analogues. Such modifications are common to those skilled in the art; a summary of nucleic acids modified in representative manners and generally referred to as nucleic acid analogues and the principle of action thereof are described in the article by Zon (1988).

With regard to the size of the nucleic acids the invention also allows a wide range. There is no theoretical upper limit imposed by the conjugates according to the invention, provided that the antibody-polycation/nucleic acid complexes are assured of being conveyed into the cells. Any lower limit is a result of reasons specific to the particular application e.g. because antisense oligonucleotides of less than about 10 nucleotides cannot be used on the grounds of insufficient specificity. Using the conjugates according to the invention plasmids can also be conveyed into the cells. Smaller nucleic acids, e.g. for antisense applications, optionally in tandem, may also be used as integral components of larger gene constructs by which they are transcribed in the cell.

It is also possible to convey different nucleic acids into the cells at the same time by means of the conjugates according to the invention.

Examples of nucleic acids with an inhibiting effect are the antisense oligonucleotides mentioned above or ribozymes with a virus-inhibiting effect on the grounds of complementarity to the gene sections essential for virus replication.

The preferred nucleic acid component of the antibody-polycation-nucleic acid complexes according to the invention having an inhibiting effect on the grounds of complementarity is antisense DNA, antisense RNA or a ribozyme or the gene coding therefor. When using ribozymes and antisense RNAs it is particularly advantageous to use the genes coding therefor, optionally together with a carrier gene. By introducing the gene into the cell a considerable amplification of the RNA is achieved, compared with the introduction of RNA as such, and consequently a supply which is sufficient for the intended inhibition of biological reaction is assured. Particularly suitable carrier genes are the transcription units required for transcription by polymerase III, e.g. tRNA genes. Ribozyme genes, for example, may be inserted into them in such a way that when transcription is carried out the ribozyme is part of a compact polymerase III transcript. Suitable genetic units containing a ribozyme gene and a carrier gene transcribed by polymerase III are disclosed in European Patent Application A1 0 387 775. With the aid of the transport system according to the present invention the effect of these genetic units can be intensified, by ensuring an increased initial concentration of the gene in the cell.

In principle all sequences of the HIV gene the blocking of which causes the inhibition of viral replication and expression are suitable as target sequences for the construction of complementary antisense oligonucleotides or ribozymes or the genes coding therefor which can be used in the treatment of AIDS. Target sequences of primary importance are the sequences with a regulatory function, particularly of the tat-, rev- or nef-genes. Other suitable sequences are the initiation, polyadenylation, splicing tRNA primer binding site (PBS) of the LTR sequence or the tar-sequence.

Apart from nucleic acid molecules which inhibit as a result of being complementary to viral genes, it is also possible to use genes with a different mechanism of activity, e.g. those which code for virus proteins containing so-called transdominant mutations (Herskowitz, 1987). The expression of the gene products in the cell results in proteins which, in their function, dominate the corresponding wild type virus protein, as a result of which the latter cannot perform its usual function for virus replication and the virus replication is effectively inhibited. Basically, transdominant mutants of virus proteins which are necessary for replication and expression, e.g. gag-, tat- and rev-mutants, which have been shown to have an inhibiting effect on HIV-replication (Trono et al., 1989; Green et al., 1989; Malim et al., 1989) are suitable.

Other examples of therapeutically active nucleic acids are those with an inhibitory effect on oncogenes.

With the aid of the present invention it is also possible to transport genes or sections thereof into the cell, the expression products of which perform a function in the transmission of signals in order to have a positive influence on signal transmission into the target cells, e.g. CD4+ cells, more particular T-cells, and thereby, for example, increase the survival of T-cells.

Theoretically, all genes or gene sections which have a therapeutic or gene-therapeutic effect in cells which express a cell surface protein are suitable for the purposes of the present invention.

Examples of genes which may be used in gene therapy and introduced into the cell by means of the present invention are factor VIII (e.g. Wood et al., 1984), factor IX (used in haemophilia; e.g. Kurachi and Davie, 1982), adenosine deaminase (SCID; e.g. Valerio et al., 1984), α-1-antitrypsin (lung emphysema; e.g. Ciliberto et al., 1985) or the “cystic fibrosis transmembrane conductance regulator gene” (Riordan et al., 1989). These examples do not constitute any kind of restriction.

The ratio of nucleic acid to conjugate may vary within wide limits and it is not absolutely necessary to neutralise all the charges of the nucleic acid. This ratio will have to be adjusted for each individual case in accordance with criteria such as the size and structure of the nucleic acid to be transported, the size of the polycation, the number and distribution of its charges, so that there is a favourable ratio, for the particular application, between the transportability and biological activity of the nucleic acid. This ratio can initially be coursely adjusted, perhaps by means of the delay in the speed of migration of the DNA in a gel (e.g. by means of mobility shift on an agarose gel) or by density gradient centrifugation. After this preliminary ratio has been obtained it may be advisable to carry out transport tests with the radioactively labelled complex with a view to obtaining the maximum available activity of the nucleic acid in the cell and possibly reducing the conjugate portion so that the remaining negative charges of the nucleic acid do not impede transport into the cell.

The preparation of the antibody-polycation/nucleic acid complexes may be carried out by methods known per se for the complexing of polyionic compounds. One possible way of avoiding uncontrolled aggregation or precipitation consists in mixing the two components at a high dilution (≦100 μg).

The antibody-polycation-nucleic acid complexes which can be absorbed into higher eukaryotic cells by endocytosis may additionally contain one or more polycations in a non-covalently bound form which may be identical to the polycation in the conjugate, so as to increase the internalisation and/or expression of the nucleic acid achieved by means of the conjugate.

With the aid of such measures, which are the subject matter of the unpublished International Patent Application No. 92/00217, a smaller amount of antibody-polycation conjugate is required, based on the quantity of nucleic acid to be imported into the cell, to achieve at least the same efficiency of transfection/expression, which means on the one hand that synthesis is less costly. A smaller amount of conjugate may also be advantageous when it is desirable to avoid the effect of having several adjacent “docking sites” occupied by a large number of antibody molecules within a complex, with the consequence that they are no longer available for additional complexes. Restricting the quantity of antibody contained in the complexes to the necessary minimum, i.e. keeping the quantity of conjugate as small as possible and diluting it with free polycation, is particularly advantageous when there is only a small number of cell surface proteins on the target cells to be treated.

With the aid of such measures, the performance of conjugates which are not particularly efficient per se can be increased substantially and the performance of conjugates which are already highly efficient can be increased still further.

With regard to the qualitative composition of the complexes according to the invention, first of all the nucleic acid to be imported into the cell and the antibody are generally determined. The nucleic acid is defined primarily by the biological effect to be achieved in the cell, e.g. by the target sequence of the gene or gene section to be inhibited or (when used in gene therapy) to be expressed, e.g. in order to substitute a defective gene. The nucleic acid may optionally be modified, e.g. because of the need for stability for the particular application. Starting from the determination of nucleic acid and antibody the polycation is matched to these parameters, the size of the nucleic acid being of critical importance, particularly with regard to the substantial neutralisation of the negative charges.

When choosing the non-covalently bound polycations which may be contained in the complexes, it is crucial that the addition of these substances should bring about an increase in the internalisation/expression of the nucleic acid, compared with that which can be achieved by means of the conjugates.

Like the qualitative composition, the quantitative composition of the complexes is also determined by numerous criteria which are functionally connected with one another, e.g. whether and to what extent it is necessary or desirable to condense the nucleic acid, what charge the total complex should have, to what extent there is a binding and internalising capacity for the particular type of cell and to what extent it is desirable or necessary to increase it. Other parameters for the composition of the complex are the accessibility of the antibody for the cell surface protein, the crucial factor being the way in which the antibody is presented within the complex relative to the cell. Another essential feature is the accessibility of the nucleic acid in the cell in order to perform its designated function.

The polycations contained in non-covalently bound form in the complexes may be the same as or different from those contained in the conjugate. An essential criterion for selecting them is the size of the nucleic acid, particularly with respect to the condensation thereof; with smaller nucleic acid molecules, compacting is not generally required. The choice of the polycations, in terms of the nature and quantity thereof, is also made in accordance with the conjugate, particular account being taken of the polycation contained in the conjugate: if for example the polycation is a substance which has no or very little capacity for DNA condensation, it is generally advisable, for the purpose of achieving efficient internalising of the complexes, to use those polycations which possess this quality to a greater extent. If the polycation contained in the conjugate is itself a substance which condenses nucleic acid and if adequate compacting of the nucleic acid for efficient internalisation is achieved, it is advisable to use a polycation which brings about an increase in expression by other mechanisms.

What is essential for the non-covalently bound polycation which may optionally also be contained in the complex is its ability to condense nucleic acid and/or to protect the latter from undesirable breakdown in the cell. The invention further relates to a process for introducing nucleic acid or acids into human or animal cells, in which preferably an antibody-polycation/nucleic acid complex which is soluble under physiological conditions is brought into contact with the cells.

Within the scope of the present invention, the DNA component used was the luciferase gene as a reporter gene (on the basis of results obtained in preliminary tests with transferrin-polycation/DNA complexes in which the luciferase gene was used as a reporter gene, it had been shown that the efficiency of import of the luciferase gene could indicate the usefulness of other nucleic acids and the nucleic acid used, in qualitative terms, is not a limiting factor for the use of protein-polycation DNA complexes.

For certain embodiments of the present invention it may be useful to create conditions under which the degradation of the nucleic acid in the cells is inhibited or prevented.

Conditions under which the breakdown of nucleic acids is inhibited may be provided by the addition of so-called lysosomatropic substances. These substances are known to inhibit the activity of proteases and nucleases in lysosomes and are thus able to prevent the degradation of nucleic acids (Luthmann & Magnusson, 1983).

These substances include chloroquin, monensin, nigericin, ammonium chloride and methylamine.

The necessity of using a substance selected from the group of lysosomatropic substances within the scope of the invention will depend in particular on the type of cell to be treated, or if different antibodies are used, it will depend on different mechanisms by which the complexes are absorbed into the cell. Thus, for example, within the scope of the present invention, it was found that the import of DNA into the cell was differently affected by chloroquin when different antibodies were used (monoclonal anti-CD4 antibodies).

In any case, it is necessary to test the necessity for or suitability of such substances within the scope of the present invention by means of preliminary trials.

The invention further relates to pharmaceutical compositions containing as active component one or more therapeutically or gene therapeutically active nucleic acids complexed with an antibody-polycation conjugate (antibody-polycation conjugate and nucleic acid may also occur separately and be complexed immediately before therapeutic use). Any pharmaceutically acceptable carrier, e.g. saline solution, phosphate-buffered saline solution, or other carriers in which the compositions according to the invention have the required solubility characteristics may be used. For formulations, reference is made to Remington's Pharmaceutical Sciences, 1980.

Examples of therapeutically active nucleic acids include the antisense oligonucleotides or ribozymes mentioned hereinbefore or the genes coding for them or genes coding for transdominant mutants, which have an inhibiting effect on endogenous or exogenous genes or gene products contained in the particular target cells. These include, for example, those genes which, by virtue of their sequence specificity (complementarity to target sequences, coding for transdominant mutants (Herskowitz, 1987)), bring about an intracellular immunity. (Baltimore, 1988) against HIV and can be used in the treatment of the AIDS syndrome or to prevent activation of the virus after infection.

The pharmaceutical preparations may be used to inhibit viral sequences, e.g. HIV or related retroviruses in the human or animal body. An example of therapeutic application by inhibiting a related retrovirus is the treatment of proliferative T-cell leukaemia which is caused by the HTLV-1 virus.

In addition to the treatment of viral T-cell leukaemias the present invention may also be used for treating non-viral leukaemias. Recently the involvement of oncogenes (abl, bcr, Ha, Ki, ras, rat, c-myc, N-myc) in the formation of lymphatic leukaemias has been demonstrated; it is thought probable that there are other oncogenes, on the basis of observed specific chromosome translocations. Cloning of these oncogenes forms the basis for the construction of oncogene-inhibiting nucleic acid molecules and hence for a further possible therapeutic use of the present invention.

Another important field of use is gene therapy. In theory, in the scope of gene therapy by means of the present invention it is possible to use all those genes or sections thereof introduced into the target cells, the expression of which produces a therapeutic effect in this type of cell, e.g. by substituting genetically caused defects or by triggering an immune response.

For therapeutic use the pharmaceutical preparation may be administered systemically, e.g. intravenously. The target tissues may be the lungs, spleen, bone marrow and tumours.

Examples of local use are the lungs (use of the pharmaceutical preparations according to the invention for instillation or as an aerosol for inhalation), direct injection into the muscle tissue, into a tumour or into the liver, or local application in the gastrointestinal tract or in sections of blood vessel.

The substances may also be administered therapeutically ex vivo, where the treated cells, e.g. bone marrow cells or hepatocytes, are reintroduced into the body (e.g. Ponder et al., 1991).

### SUMMARY OF THE FIGURES

FIG. 1: Introduction of antiCD4-polylysine/pRSVL complexes into CD4⁺-CHO cells

FIG. 2: Import of antiCD4-polylysine/pRSVL complexes into CD4⁺-CHO cells

FIGS. 3,4 Transfer and expression of DNA in H9-cells by means of antiCD7-polylysine 190 conjugates

FIG. 5: Transfer of DNA into pancreas carcinoma cells by means of mAb1.1ASML-polylysine 190 conjugates

FIG. 6: Transfer of DNA into CD4⁺ cells using antibody protein A-polylysine conjugates

FIG. 7: Transfer of DNA into K562 cells with antibody-protein A/G-polylysine conjugates

The invention is illustrated by means of the Examples which follow.

### EXAMPLE 1

### Preparation of AntiCD4-Polylysine 90 Conjugates

Coupling was carried out analogously to methods known from the literature by introducing disulphide bridges after modification with succinimidyl-pyridyldithiopropionate (SPDP, Jung et al., 1981).

A solution of 1.7 mg of antiCD4 antibody (OKT4A, Ortho Diagnostic Systems) in 50 mM sodium phosphate buffer pH 7.8 was mixed with 11 μl of 10 mM ethanolic solution of SPDP (Pharmacia). After 1 hour at ambient temperature the mixture was filtered through a Sephadex G 25 gel column (eluant 100 mM HEPES buffer pH 7.3), to obtain 1.4 mg of anti-CD4, modified with 75 nmol of pyridyldithiopropionate groups. Poly(L)lysine 90 (average degree of polymerisation of 90 lysine groups (Sigma), fluorescent-labelled by means of FITC) was modified analogously with SPDP and brought into the form modified with free mercapto groups by treating with dithiothreitol and subsequent gel filtration. A solution of 38 nmol polylysine 90, modified with 120 nmol mercapto groups, in 0.5 ml of 20 mM sodium acetate buffer, was mixed with the above-mentioned modified antiCD4 with the exclusion of oxygen and left to stand overnight at ambient temperature. The conjugates were isolated by gel permeation chromatography (Superose 12, 500 mM guanidinium hydrochloride pH 7.3); after dialysis against 25 mM HEPES pH 7.3, corresponding conjugates were obtained consisting of 1.1 mg antiCD4 antibody modified with 11 nmol polylysine 90.

### EXAMPLE 2

### Preparation of AntiCD4-Polylysine 190 Conjugates

A solution of 1.0 mg (6.25 nmol) of antiCD4 antibody (OKT4A, Ortho Diagnostic Systems) in 0.3 ml of 50 mM HEPES pH 7.8 was mixed with 37 μl of 1 mM ethanolic solution of succinimidyl-pyridyldithio-propionate (SPDP, Pharmacia). After 1 hour at ambient temperature the mixture was filtered over a Sephadex G 25 column (eluant 100 mM HEPES buffer pH 7.9), to obtain 0.85 mg (5.3 nmol) of antiCD4 modified with 30 nmol pyridyldithiopropionate groups. Poly(L)lysine190 (average degree of polymersation of 190 lysine groups (Sigma), fluorescent-labelled by means of FITC) was modified analogously with SPDP and brought into the form modified with free mercapto groups by treating with dithiothreitol and subsequent gel filtration. A solution of 7.7 nmol of polylysine 190, modified with 25 nmol of mercapto groups, in 0.13 ml of 30 mM sodium acetate buffer was mixed with the above-mentioned modified antiCD4 (in 0.5 ml of 300 mM HEPES pH 7.9) with the exclusion of oxygen and left to stand overnight at ambient temperature. The reaction mixture was adjusted to a content of about 0.6 M by the addition of 5 M NaCl. The conjugates were isolated by ion exchange chromatography (Mono S, Pharmacia, 50 mM HEPES pH 7.3, salt gradient 0.6 M to 3 M NaCl); after dialysis against 10 mM HEPES pH 7.3, corresponding conjugates were obtained consisting of 0.35 mg (2.2 nmol) of antiCD4 antibody, modified with 3.9 nmol of polylysine 190.

### EXAMPLE 3

### Preparation of AntiCD7-Polylysine 190 Conjugates

A solution of 1.3 mg of antiCD7 antibody (Immunotech) in 50 mM HEPES pH 7.9 was mixed with 49 μl of 1 mM ethanolic solution of SPDP (Pharmacia). After 1 hour at ambient temperature the mixture was filtered over a Sephadex G 25 gel column (eluant 50 mM HEPES buffer 7.9), to obtain 1.19 mg (7.5 nmol) of antiCD7, modified with 33 nmol of pyridyldithiopropionate groups. Poly(L)lysine 190, fluorescent labelled by means of FITC, was modified analogously with SPDP and brought into the form modified with free mercapto groups by treatment with dithiothreitol and subsequent gel filtration.

A solution of 11 nmol of polylysine 190, modified with 35 nmol mercapto groups, in 0.2 ml of 30 mM sodium acetate buffer was mixed with the above-mentioned modified antiCD7 (in 0.5 ml of 300 mM HEPES pH 7.9) with the exclusion of oxygen and left to stand overnight at ambient temperature. The reaction mixture was adjusted, by the addition of 5 M NaCl, to a content of about 0.6 M. The conjugates were isolated by ion exchange chromatography (Mono S, Pharmacia, 50 mM HEPES pH 7.3, salt gradient 0.6 M to 3 M NaCl); after dialysis against 10 mM HEPES pH 7.3, corresponding conjugates were obtained, consisting of 0.51 mg (3.2 nmol) of antiCD7 antibody, modified with 6.2 nmol of polylysine 190.

### EXAMPLE 4

### Preparation of mAb1.1ASML-Polylysine 190 Conjugates

In this Example a monoclonal antibody against the membrane protein preparation of the rat pancreas carcinoma cell line BSp73ASML (Matzku et al, 1983) designated mAb1.1ASML was used for the preparation of the conjugates. A solution of 2.0 mg of mAb1.1ASML in 0.5 ml of 50 mM HEPES pH 7.9 was mixed with 75 μl of 1 mM ethanolic solution of SPDP (Pharmacia). After 1 hour at ambient temperature the mixture was filtered over a Sephadex G 25 gel column (eluant 50 mM HEPES buffer pH 7.9), to obtain 1.3 mg (8 nmol) of mAb1.1ASML, modified with 39 nmol of pyridyldithiopropionate groups. Poly(L)lysine 190, fluorescent-labelled by means of FITC, was modified analogously with SPDP and brought into the form modified with free mercapto groups by treatment with dithiothreitol and subsequent gel filtration. A solution of 12 nmol of polylysine 190, modified with 37 nmol of mercapto groups in 210 μl of 30 mM sodium acetate buffer was mixed with the above-mentioned modified mAb1.1ASML (in 0.9 ml of 100 mM HEPES pH 7.9) with the exclusion of oxygen and left to stand overnight at ambient temperature. The reaction mixture was adjusted to a content of about 0.6 M by the addition of 5 M NaCl. The conjugates were isolated by ion exchange chromatography (Mono S, Pharmacia, 50 mM HEPES pH 7.3, saline gradient 0.6 M to 3 M NaCl); after fractionation and dialysis against 20 mM HEPES pH 7.3, conjugate fractions A consisting of 0.16 mg (1 nmol) of mAb1.1ASML, modified with 0.45 nmol of polylysine 190 was obtained (in the case of fraction A), 0.23 mg (14 nmol) of mAb1.1ASML, modified with 0.9 nmol of polylysine 190 (fraction B), or 0.92 mg (5.8 nmol) of mAb1.1ASML, modified with 3.9 nmol of polylysine 190 (fraction C) were obtained.

### EXAMPLE 5

### Preparation of Protein-A Polylysine 190 Conjugates

A solution of 4.5 mg of protein-A (Pierce, No. 21182, 107 nmol) in 0.5 ml of 100 mM HEPES pH 7.9 was mixed with 30 μl of 10 mM ethanolic solution of SPDP (Pharmacia). After 2 hours at ambient temperature the mixture was filtered over a Sephadex G25 gel column (eluant 50 mM HEPES buffer pH 7.9) to obtain 3.95 mg (94 nmol) of protein-A, modified with 245 nmol of pyridyldithiopropionate groups. Poly(L)lysine 190, fluorescent-labelled by means of FITC, was modified analogously with SPDP and, by treatment with dithiothreitol and subsequent gel filtration, brought into the form modified with free mercapto groups. A solution of 53 nmol of polylysine 190, modified with 150 nmol of mercapto groups, in 0.8 ml of 30 mM sodium adetate buffer was mixed with the above-mentioned modified protein-A, with the exclusion of oxygen, and left to stand overnight at ambient temperature. The reaction mixture was adjusted to a content of approximately 0.6 M by the addition of 5 M NaCl. The conjugates were isolated by ion exchange chromatography (Mono S, Pharmacia, 50 mM HEPES pH 7.3, salt gradient 0.6 M to 3 M NaCl); after fractionation and dialysis against 25 mM HEPES-pH 7.3, two conjugate fractions A and B were obtained, consisting of 1.15 mg (27 nmol) of protein A, modified with 5 nmol of polylysine 190 (in the case of fraction A) and 2.6 mg (6.2 nmol) of protein-A, modified with 40 nmol of polylysine 190 (fraction B).

### EXAMPLE 6

### Preparation of Complexes of Antibody-Polycation Conjugates with DNA

The complexes were prepared by mixing dilute solutions of DNA (30 μg/ml or less in 150 mM NaCl, 20 mM HEPES pH 7.3) with the antibody-polylysine conjugates obtained in Examples 1, 2 and 4 (1.00 μg/ml or less). The DNA used was PRSVL plasmid DNA (De Wet et al., 1987) prepared by Triton-X lysis standard method (Maniatis, 1982) followed by CsCl/EtBr equilibrium density gradient centrifugation, decolorising with butanol-1 and dialysis against 10 mM Tris/HCl pH 7.5, 1 mM EDTA. In order to prevent precipitation of the DNA complexes, phosphate-free buffer was used (phosphates decrease the solubility of the conjugates).

### EXAMPLE 7

### Transfer and Expression of DNA in CD4⁺ CHO-Cells by Means of AntiCD4-Polylysine 90 Conjugates

In this and the following Examples plasmid DNA containing the Photinus pyralis luciferase gene as reporter gene was used to investigate gene transfer and expression. In the Figures which show the results of the experiments, the values given for the luciferase activity relate to the activity of the entire cell sample.

CD4⁺ CHO-cells (Lasky et al., 1987) were seeded, at a rate of 5×10⁵ cells per T-25 vial, in Ham's F-12 medium C, (Ham, 1965) plus 10% FCS (foetal calves' serum). 18 hours later the cells were washed twice with Ham's F-12 medium without serum and incubated in this medium (5 ml) for 5 hours at 37° C.

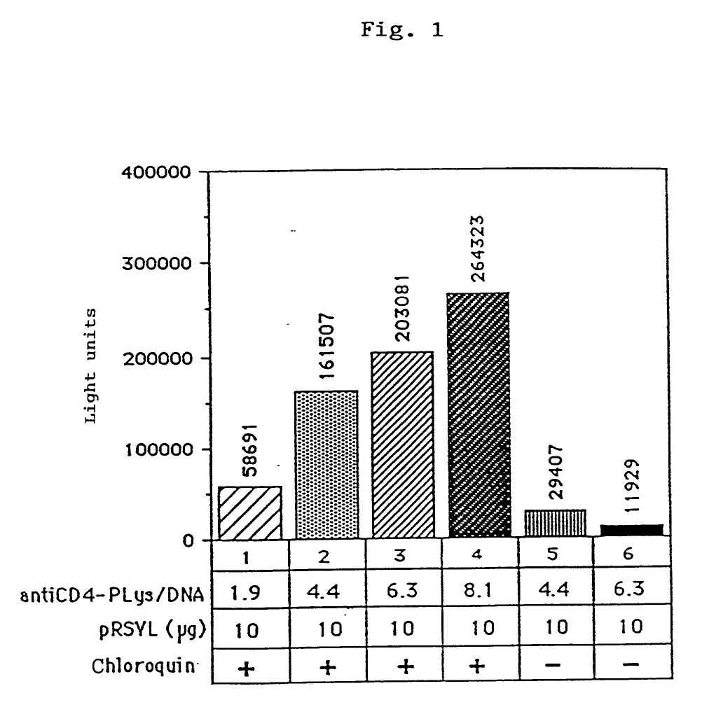

Anti-CD4 polylysine/pRSVL complexes were prepared at final concentrations of DNA of 10 μg/500 μl in 150 mM NaCl, 20 mM HEPES pH 7.5, as described in Example 6. Anti-CD4 polylysine 90 (8.4 nmol polylysine 90/mg anti-CD4) were used in the mass ratios specified (from 1.9 to 8.1 expressed as mass of anti-CD4). In samples 1 to 4 the complexes were added to the cells in Ham's F-12 medium without serum, containing 100 μM chloroquin; in samples 5 and 6 the chloroquin was omitted. After 4 hours' incubation the cells were washed twice with medium plus 10% FCS and incubated in this medium. In samples 5 and 6 the same volume of serum-containing medium was added to the cells. After 20 hours all the cells were washed with fresh serum-containing medium and harvested 48 hours later. Aliquots of extracts (about ⅕ of each sample, corresponding to the same amount of protein, were investigated for lucerifase activity (De Wet et al., 1987). The bioluminescence was measured using clinilumate (Berthold, Wildbach, FRG). The result of these investigations is shown in FIG. 1. It was found that DNA is imported into CD4⁺ cells by means of the conjugates according to the invention and the imported DNA is expressed, the efficiency of the DNA import being proportional to the content of anti-CD4/polylysine.

### EXAMPLE 8

### Transfer and Expression of DNA Into CD4⁺ CHO Cells by Means of AntiCD4-Polylysine 190 Conjugates

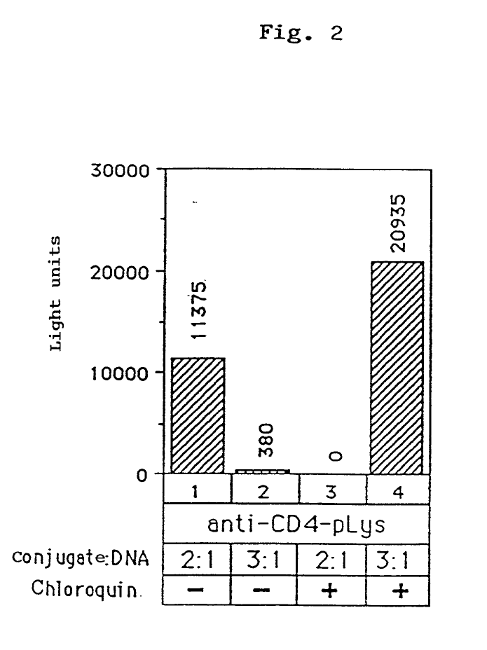

First, CD4⁺ CHO cells were cultivated as described in Example 7. Conjugate/DNA complexes, prepared as in Example 6, containing 10 μg PRSVL and either a 2:1 or 3:1 mass excess of antiCD4-polylysine 90 (see Example 1), as stated in FIG. 2, were added to the cells in the absence or presence of 100 μM chloroquin. After a further 4 hours at 37° C. the samples containing chlaroquin were washed twice with Ham's medium, containing 10% foetal calves' serum, whilst 5 ml of the same medium were added to the samples containing no chloroquin. The cells were incubated for a further 20 hours at 37° C. and aliquots were investigated for their luciferase activity, as stated in Example 7. The results of these tests are shown in FIG. 2.

### EXAMPLE 9

### Transfer and Expression of DNA in H9-Cells By Means of AntiCD7-Polylysine 190 Conjugates

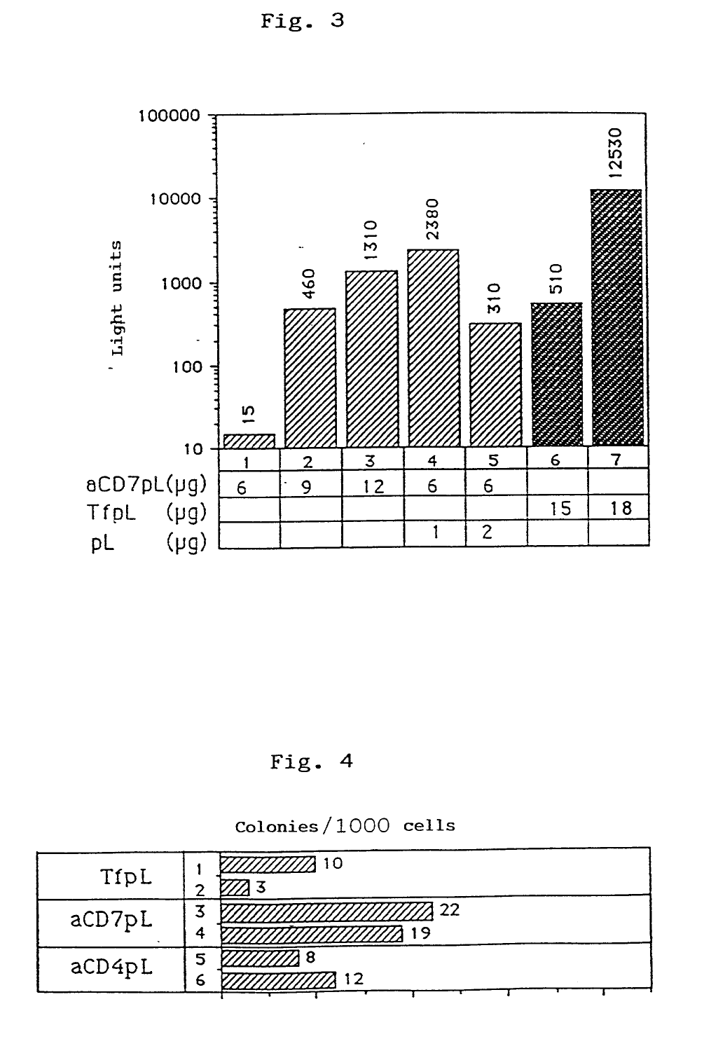

a) Cells of the T-cell line H9 (Mann et al., 1989) were cultivated in RPMI 1640 medium, supplemented with 20% FCS, 100 units per ml of penicillin, 100 μg/ml of streptomycin and 1 mM glutamine. Immediately before transfection the cells were collected by centrifuging and taken up in fresh medium at the rate of 100,000 cells per ml (1,000,000 cells per sample), which were used for transfection. As a comparison with antiCD7 conjugates, transferrin conjugates were used. Transferrin-polylysine conjugates were prepared as described in EP-A 1 388 758; the antiCD7 conjugates used were those described in Example 3. Complexing with DNA was carried out as stated in Example 6. For transient transfection in H9 cells the DNA used was the plasmid pHLuci which contains the HIV-LTR sequence combined with the sequence which codes for luciferase, followed by the SV40-intron/polyA site: the HindIII fragment containing the protease 2A gene from pHIV/2A (Sun and Baltimore, 1989) was removed and replaced by a HindIII/SmaI fragment of pRSVL (De Wet et al., 1987) containing the sequence which codes for luciferase. The two fragments were joined via the HindIII sites (after smooth ends had been produced using Klenow fragment) and then linked via the smooth SmaI site to the now smooth HindIII site. A clone having the correct orientation of the luciferase gene sequence was selected. This plasmid requires the TAT gene product for a strong transcription activity. This is prepared by co-transfection with the plasmid pCMVTAT, which codes for the HIV-TAT gene under the control of the CMV immediate early promoter (Jakobovits et al., 1990). The DNA complexes used for transfection contain a mixture of 5 μg of pHLuci and 1 μg of pCMVTAT. The DNA/polycation complexes (500 μl) were added to the 10 ml cell sample and incubated for 4 hours in the presence of 100 μM chloroquin. Then the cells were washed in fresh medium, harvested 40 hours later and investigated for their luciferase activity as described in the preceding Examples. The results (in luciferase light units) are given in FIG. 3: it was found that the luciferase activity increases as the amount of antiCD7-polylysine conjugate complexed with 6 μg of DNA increases (samples 1, 2 and 3). A further increase in activity was observed when 6 μg of conjugate were used together with 1 μg of free polylysine for complex formation (sample 4), whilst a further addition of polylysine affected the gene transport (sample 5). (The comparison tests carried out with transferrin-polylysine conjugates are designated 6 and 7.)

b) A further series of tests for transfection using the antibody conjugates was carried out using the plasmid PSSTNEO. This plasmid, which contains a neomycin resistance gene as marker, was introduced into H9 cells using antiCD4, antiCD7 and (for comparison) transferrin-polylysine 190 conjugates (6 μg of DNA were used per 10⁶ cells; the optimum transfection conditions had been determined in preliminary trials using transient luciferase assays). The plasmid pSSTNEO contains the large Sst fragment of the pUCu locus (Collis et al., 1990) which contains the HSV TK-neo unit. A 63 bp fragment containing a single NdeI site had been introduced into the Asp718 site. Aliquots of the transfected cells (containing a defined number of cells) were then diluted in a semisolid methylcellulose medium containing 1000 μg/ml G418. In order to do this, aliquots of the cells were plated out 3 days after transfection with DNA, containing the neomycin marker, in a semisolid medium which contained in addition to the normal requirements 0.5-1 mg/ml of G418 and 20 mg/ml of methylcellulose. (In order to prepare the semisolid selection medium a solution of 20 g of methylcellulose in 460 ml of water was prepared under sterile conditions.) Then 500 ml of doubly concentrated, supplemented nutrient medium, also prepared under sterile conditions, were combined with the methylcellulose solution, the volume was adjusted to 1 litre and the medium was stirred overnight at 4° C. 50 ml aliquots of this medium were mixed with 10 ml of serum, optionally after storage at −20° C., and the volume was adjusted to 100 ml with complete medium containing no serum. At this stage G418 was added. A 2.5 ml aliquot of the methylcellulose medium was mixed with a 50 to 100 μl aliquot of the cell suspension and about 1 ml of this mixture was poured into culture dishes. Incubation was carried out at 37° C. under a CO₂ atmosphere. About 10 to 14 days later the G418-resistant cells were counted (only colonies containing more than 200 cells were counted as positive). The results are shown in FIG. 4 (this shows the number of G418-resistant colonies per 1000 cells 10 days after being placed in the antibiotic medium).

### EXAMPLE 10

### Transfer of DNA Into Pancreas Carcinoma Cells By Means of mAb1.1ASML-Polylysine 190 Conjugates

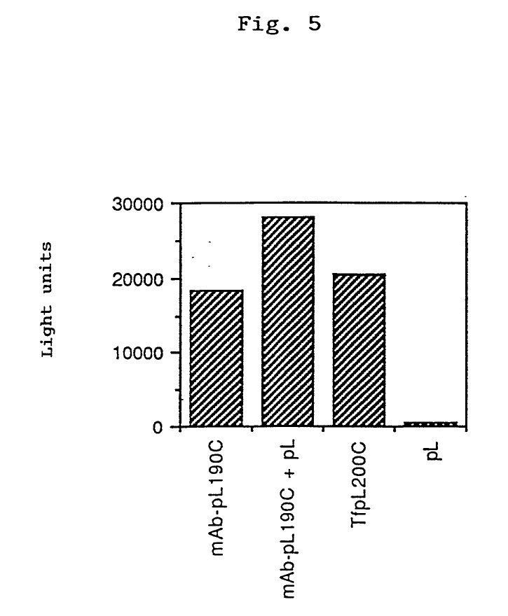

Cells of the metastasising rat pancreas carcinoma cell line-BSp73ASML (Matzku et al., 1989) in 2 ml of RPMI 1640 medium-plus 10% FCS were plated out at the rate of 5×10⁵ cells in 24-well plates made by Falcon. The cells were brought into contact with the mAb1.1ASML-polylysine 190 conjugates prepared in Example 4 (or as a comparison with transferrin-polylysine 200 conjugates or with polylysine on its own), complexed with pRSVL-DNA, in the ratios of conjugate to DNA specified below, in the presence of 100 μM of chloroquin. After 4 hours incubation at 37° C. with the complexes, the medium was eliminated, fresh serum-containing medium (without chloroquin) was added and the cells were harvested after 20 hours at 37° C. From the cell extracts, aliquots standardised for a similar protein content were investigated for luciferase activity. The values for light units given in FIG. 5 correspond to the luciferase activity of 5×10⁵ cells transfected with 6 μg DNA (in the Figure mAb-pL190C denotes 18 μg of mAb1.1ASML-pL190C conjugate; mAb-pL190C+pL denotes 9 μg of mAb1.1ASMLpL-190C conjugate+1.5 μg of non-conjugated poly(L)lysine 90; TfpL200 denotes 18 μg TfpL200C (transferrin-polylysine 200 conjugate) and pL denotes 2.5 μg (or 1-4 μg) of poly(L)lysine 90).

### EXAMPLE 11

### Transfection of CD4⁺ Cells With Antibody Protein A-Polylysine Conjugates

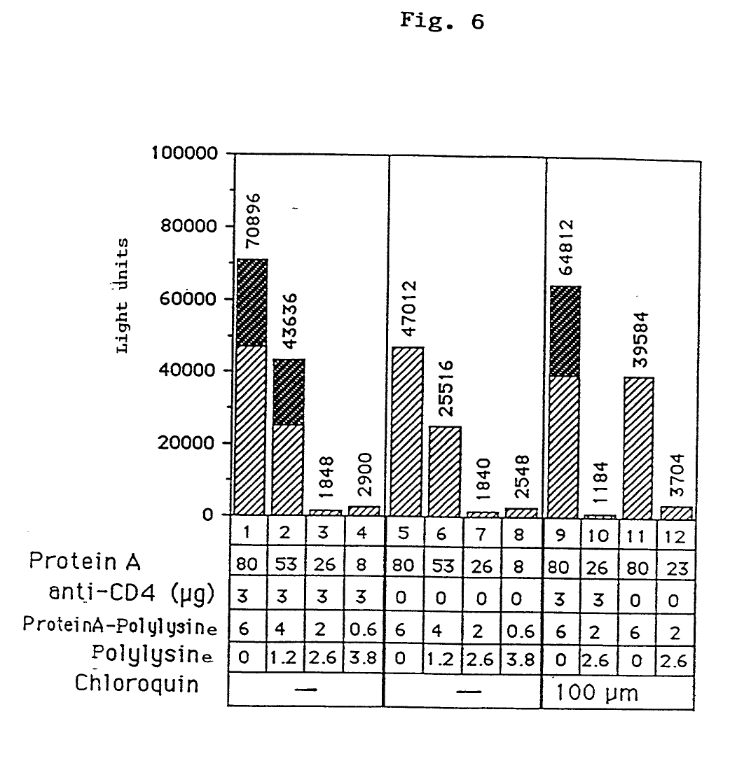

CD4-expressing HeLa cells (see Example 7) were seeded at the rate of 6×10⁵ cells per T25 vial and then grown in DME medium plus 10% FCS. Where shown in FIG. 6, the cells were pre-incubated with the antibody (anti-CD4gp55kD, IOT4, Immunotech) (3 μg per sample) for 1 hour at ambient temperature. In the meantime, protein A-polylysine 190/DNA complexes were prepared in 500 μl of HBS, containing 6 μg of PRSVL and the specified amounts of protein A-polylysine 190 plus additional free polylysine as in Example 6. After the end of the 1 hour incubation the cells were placed in 4.5 μl of fresh medium and the 500 μl DNA sample was added to the cells at 37° C. After 4 hours, those samples which contained 100 μM chlorpquin (samples 9-12) were washed in fresh medium, whilst samples 1-8 were incubated until harvesting with the DNA. For the luciferase assay the cells were harvested 20 hours later. The results of the experiments are shown in FIG. 6. It was found that the luciferase activity was dependent on the presence of protein A-polylysine in the DNA complex (samples 1-4, 5, 6). In samples 5-8, 11, 12 there was evidence of DNA transportation by means of the protein A complex without any antibody pretreatment; however, the introduction of DNA was increased by about 30% when the cells had been pretreated with the antibody which recognises the cell surface protein CD4 (samples 1-4, 9, 10). It was also found that the presence of chloroquin does not cause an increase in DNA expression (cf. samples 1-8 with samples 9-12).

### EXAMPLE 12

### Transfection of K562 Cells With Antibody-Protein A/G Polylysine 190 Conjugates

a) Preparation of Protein A/G Polylysine 190 Conjugates

A solution of 4.5 mg of recombinant protein A/G (Pierce, No. 21186, 102 nmol) in 0.5 ml of 100 mM HEPES pH 7.9 was mixed with 30 μl of 10 mM ethanolic solution of SPDP (Pharmacia). After 2 hours at ambient temperature the mixture was filtered over a Sephadex G 25 gel column (eluant 50 mM HEPES buffer pH 7.9), to obtain 3.45 mg (75 nmol) of protein A/G, modified with 290 nmol of pyridyldithiopropionate groups. Poly(L)lysine 190, fluorescent-labelled by FITC, was modified analogously with SPDP and brought into the form modified with free mercapto groups by treatment with dithiothreitol and subsequent gel filtration.

A solution of 42 nmol of polylysine 190, modified with 130 nmol of mercapto groups, in 0.8 ml of 30 mM sodium acetate buffer was mixed with the above-mentioned modified protein A/G with the exclusion of oxygen and left to stand overnight at ambient temperature. The reaction mixture was adjusted to a content of approximately 0.6 M by the addition of 5 M NaCl. The conjugates were isolated by ion exchange chromatography (Mono S, Pharmacia, 50 mM HEPES pH 7.3, salt gradient 0.6 M to 3 M NaCl); after fractionation and dialysis against 25 mM HEPES pH 7.3 a conjugate fraction was obtained, consisting of 1.02 mg (22 nmol) of protein A/G, modified with 12 nmol of polylysine 190.

b) Preparation of Antibody-Protein A/G-Polylysine 190/DNA Complexes

The solution of 7 μg of the protein A/G conjugate prepared in a) in HBS was bound, by mixing, to the monoclonal antibody CD7 antibody directed against the transferrin receptor (3 μg, clone BU55, IgG1, The Binding Site Limited, Birmingham, England). DNA complexes were formed by mixing a solution of the resulting antiCD71-bound protein A/G-polylysine conjugate in 200 μl HBS with a solution of 6 μg plasmid DNA (containing the luciferase gene as reporter gene, cf. the preceding Examples) in 300 μl HBS.

c) Gene Transfer Into K562 Cells

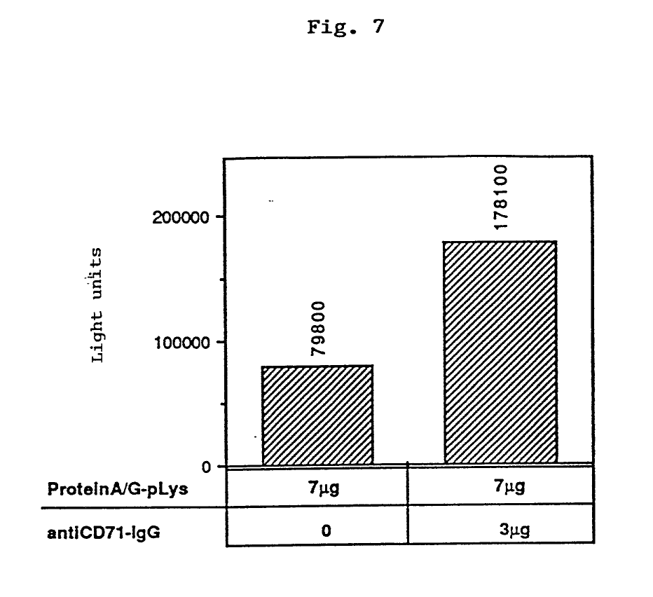

K562 cells (ATCC CCL243), which are rich in transferrin receptor, were grown in suspension in RPMI 1640 medium (Gibco BRL plus 2 g sodium bicarbonate/l) plus 10% FCS, 100 U/ml penicillin, 100 μg/ml streptomycin and 2 mM glutamine, to a density of 500,000 cells/ml. 20 hours before transfection the cells were added to fresh medium containing 50 μM deferrioxamine (Sigma). The cells were harvested, taken up in fresh medium containing 10% FCS (plus 50 μM deferrioxamine), at a rate of 250,000 cells/ml and placed in a plate having 24 wells (2 ml per well). During the first 4 hours of the experiment the medium contained 100 μM chloroquin. The cells were washed in fresh medium without chloroquin and harvested 24 hours later. The luciferase activity was determined as described in the preceding Examples. The results of the experiments are given in FIG. 7.

### BIBLIOGRAPHY

Aruffo A. and Seed B., 1987, EMBO J. 6, No. 11, 3313-3316

Baltimore D., 1988, Nature 335, 395

Chaudhary V. K. et al., 1990, Proc.Natl.Sci.USA 87, 1066

Ciliberto G. et al., 1985, Cell 41, 531-540

Co M. S. and Queen C., 1991, Nature 351, 501-502

Collis P. et al., 1990, EMBO J. 9, 233-240

De Wet et al., 1987, Mol.Cell.Biol. 7, 725-737

Green M. et al., 1989, Cell 58, 215-223

Ham R. G., 1965, Proc.Natl.Acad.Sci.USA 53, 288

Herskowitz I., 1987, Nature 329, 219

Jakobovits A. et al., 1990, EMBO J. 9, 1165-1170

Johnston S. A., 1990, Nature 346, 776-777

Jung et al., 1981, Biochem.Res.Commun.101, 599

Kasid A. et al., 1990, Proc.Natl.Acad.Sci.USA 87, 473-477

Knapp W. et al., 1989, Immunol. Today 10, 253-258

Kurachi K. and Davie E. W., 1982, Proc.Natl.Acad.Sci.USA 79, 6461-6464

Lasky et al., 1987, Cell 50, 975-985

Luthmann H. and Magnusson, 1983, Nucl.Acids Res.11 1295-1308

Malim M. et al., 1989, Cell 58, 205-214

Maniatis, 1982, Molecular Cloning, Cold Spring Harbor

Mann D. L. et al., 1989, AIDS Research and Human Retroviruses, 5, 253

Matzku S. et al., 1983, Invasion Metastasis 3, 109-123

Matzku S. et al., 1989, Cancer Res. 49, 1294-1299

Nabel E. G. et al., 1990, Science 249, 1285-1288

Orlandi R. et al., 1989, Proc.Natl.Acad.Sci.USA 86, 3833-3837

Pelchen-Matthews et al., 1989, EMBO J.8, 3641-3649

Ponder K. P. et al., 1991, Prod.Natl.Acad.Sci.USA 88, 1217-1221

Riordan J. R. et al., 1989, Science 245, 1066-1073

Sastry L. et al., 1989, Proc.Natl.Acad.Sci.USA 86, 5728-5732

Sun X.-H. and Baltimore D., 1989, Proc.Natl.Acad.Sci. USA 86, 2143-2146

Surolia A. et al., 1982, TIBS—February, 74-76

Trono D. et al., 1989, Cell 59, 113-120

Valerio D. et al., 1984, Gene 31, 147-153

Wagner E. et al., 1991, Bioconjugate Chemistry 2, 226-231

Waldmann T. A., 1991, Science 252, 1657-1662

Wilson J. M. et al., 1990, Proc.Natl.Acad.Sci.USA 87, 439-443

Wood W. I. et al., 1984, Nature 312, 330-337

Wu G. Y. and Wu C. H., 1987, J.Biol.Chem. 263, 14621-14624

Yang N. S. et al., 1990, Proc.Natl.Acad.Sci.USA 87, 9568-9572

Zamecnik et al., 1986, Proc.Natl.Acad.Sci.USA 83, 4143

Zon G., 1988, Pharmaceut. Research 5, No.9, 539-549 Remington's Pharmaceutical Sciences, Mack Publishing Co., Easton, Pa., Osol (ed.), 1980

1. New protein-polycation conjugates which are capable of forming soluble complexes with nucleic acids which are absorbed into human or animal cells, characterised in that the protein component of the conjugates is an antibody, or a fragment thereof, directed against a cell surface protein of the target cells, with the ability to bind to the cell surface protein, so that the complexes formed are absorbed in cells which express the cell surface protein.

2. Conjugates according to claim 1, characterised in that the antibody is a monoclonal antibody or a fragment thereof.

3. Conjugates according to claim 1 or 2, characterised in that the antibody is directed against a cell surface protein which is expressed on cells of the T-cell lineage.

4. Conjugates according to claim 3, characterised in that the antibody is directed against CD4.

5. Conjugates according to claim 1 or 2, characterised in that the antibody is directed against a tumour antigen.

6. Conjugates according to claim 3, characterised in that the antibody is directed against CD7.

7. Conjugates according to one of claims 1 to 6, characterised in that the antibody is coupled directly to the polycation.

8. Conjugates according to one of claims 1 to 6, characterised in that they contain an antibody which is bound via optionally modified protein A coupled to polycation.

9. Protein A-polycation conjugates for preparing antibody conjugates according to claim 8.

10. Conjugates according to one of claims 1 to 8, characterised in that the polycation is a synthetic homologous or heterologous polypeptide.

11. Conjugates according to claim 10, characterised in that the polypeptide is polylysine.

12. Conjugates according to one of claims 1 to 8, characterised in that the polycation is an optionally modified protamine.

13. Conjugates according to one of claims 1 to 8, characterised in that the polycation is an optionally modified histone.

14. Conjugates according to one of claims 10 to 13, characterised in that the polycation has about 20 to 1000 positive charges.

15. Conjugates according to one of claims 10 to 14, characterised in that the molar ratio of antibody to polycation is about 10:1 to 1:10.

16. New protein-polycation/nucleic acid complexes which are absorbed into human or animal cells, characterised in that the protein component of the conjugates is an antibody, or a fragment thereof, against a cell surface protein of the target cells, with the ability to bind to the cell surface protein, so that the complexes formed are absorbed into cells which express the cell surface protein, the antibody being bound directly to the polycation or via optionally modified protein A.

17. Complexes according to claim 16, characterised in that they contain as conjugate component one of the conjugates defined in claims 1 to 8 or 11 to 15.

18. Complexes according to claim 17, characterised in that they additionally contain a non-covalently bound polycation, which may optionally be identical to the polycation of the conjugate, so that the internalisation and/or expression of the nucleic acid achieved by means of the conjugate is increased.

19. Complexes according to one of claims 16 to 18, characterised in that they contain an inhibiting nucleic acid in the form of an antisense oligonucleotide or ribozyme or the gene coding therefor, optionally together with a carrier gene.

20. Complexes according to claim 19, characterised in that the nucleic acid is a virus-inhibiting nucleic acid.

21. Complexes according to claim 20, characterised in that they contain a nucleic acid which inhibits replication and expression of the HIV-1 virus or related retroviruses.

22. Complexes according to claim 20 or 21, characterised in that they contain a nucleic acid coding for a virus protein which has a transdominant mutation.

23. Complexes according to claim 19, characterised in that they contain an oncogene-inhibiting nucleic acid.

24. Complexes according to claim 17 or 18, characterised in that they contain a therapeutically or gene therapeutically active nucleic acid in the form of a gene or gene section.

25. Process for introducing nucleic acid into higher eukaryotic cells, in which one of the complexes defined in claims 19 to 24 preferably soluble under physiological conditions, is formed from an antibody conjugate as defined in one of claims 1 to 8 or 10 to 15 and nucleic acid or acids, optionally in the presence of non-covalently bound polycation, and cells which express the cell surface protein against which the antibody is directed are brought into contact with this complex, optionally under conditions in which the breakdown of nucleic acid in the cell is inhibited.

26. Process according to claim 25 for introducing nucleic acid into higher eukaryotic cells, in which a complex is formed from a protein A-polycation conjugate consisting of optionally modified protein A and one of the polycations defined in claims 10 to 14 and one of the nucleic acids defined in claims 16 to 24 and in the presence of an antibody directed against a cell surface protein of the target cells the complex is brought into contact with cells which express this cell surface protein, the antibody being bound to the conjugate component of the complex.

27. Process according to claim 26, characterised in that the cells are pretreated with the antibody before being brought into contact with the protein A-polycation/nucleic acid complex.

28. Pharmaceutical preparation containing as active component one or more therapeutically or gene therapeutically active nucleic acids in the form of one of the complexes defined in claims 16 to 24.
